(19) United States
(12) Patent Application Publication
(10) Pub. No.: US 2009/0024010 A1
(43) Pub. Date: Jan. 22, 2009

(54) Hand-held analysis device having different setting modes

(76) Inventors: Karl Werner, Wiesloch (DE); Gertrud Albrecht, Mannheim (DE); Rudolf Supka, Heppenheim (DE)

Correspondence Address: ROCHE DIAGNOSTICS OPERATIONS INC., 9115 Hague Road, Indianapolis, IN, 46250-0457, US

(21) Appl. No.: 12/189,459

(22) Filed: Aug. 11, 2008

Related U.S. Application Data
(63) Continuation of application No. PCT/EP2007/000487, filed on Aug. 16, 2007.

(30) Foreign Application Priority Data
Feb. 11, 2006 (EP) 06002793.5

(51) Int. Cl. A61B 5/145

(52) U.S. Cl. 600/309

(57) ABSTRACT

A handheld analysis device for examining a sample of body fluid, such as blood to analyze blood glucose, has a setting mode for changing various device settings to improve the changing of device settings. The handheld analysis device comprises a measurement unit, a microprocessor, a segment display, and an operating element. When the operating element is actuated, the device is placed in a selection mode and further actuation of the operating element, then in one of several setting modes, the user can efficiently change device settings by inputting data and/or control commands.

### REFERENCE

This application is a continuation of PCT/EP2007/0000487 filed Aug. 16, 2007 which is based on and claims priority to European Patent Application No. 06002793.5 filed Feb. 11, 2006, which are hereby incorporated by reference.

### FIELD

The disclosure relates to a handheld analysis device for examining a sample of a human or animal body fluid, comprising a measuring unit for carrying out a measurement on a sample, a microprocessor for controlling the measurement unit, a segment display for displaying measurement results, and at least one operating element via which a user can input data and/or control commands.

### BACKGROUND

Handheld analysis devices for examining a sample of human fluid are commercially available and are used, in particular, by diabetics for determining the glucose content of blood. Diabetics need to determine their blood glucose content multiple times daily and are therefore need to have a suitable handheld analysis device readily available. Therefore handheld analysis devices for determining the glucose content of blood should be as small and compact as possible.

The function of modern handheld analysis devices is not limited to measurements for examining samples. Modern handheld analysis devices can compare measurement results with limit values and alert a user, for example, of blood glucose concentration values being too high or too low by means of acoustic warning signals or remind a user to carry out a measurement at times of day that can be set. However, each additional function of this type contributes to the handling of the handheld analysis device becoming more complicated and rendering its correct operation more difficult. For example in the case of devices having a reminder function, an internal clock must be set by entering the current time of day. Subsequently, reminder times need to be programmed such that a user can be reminded to carry out a measurement. Accordingly, in devices having a warning function, limit values need to be set such that a user can be alerted, by means of a warning signal, of health risks if blood glucose concentrations reach medically precarious levels.

Since the number and size of the operating elements of small and compact handheld analysis devices is very limited due to reasons of limited space, the inputting of data and control commands for setting various device functions is tedious and time-consuming for many users. This applies even more to persons, whose manual dexterity is limited due to disease or old age.

Moreover, in handheld analysis devices having a reasonably-priced segment display for a display, there are only limited options available for simplifying the inputting of data and control commands by a user by means of suggestive visualizations and to support a user during the adaptation of various device functions for the first start-up of the device. In contrast, using a dot matrix, which is present for example in the device known from European Patent EP 1 369 688 A2, extensive options are available for supporting a user by means of complex visualizations.

### SUMMARY

Embodiments of the invention offer a way of simplifying a user's changing of his personal user profile, or a device setting.

In one embodiment of the invention, a handheld analysis device for examining a sample of a human or animal body fluid, comprises a measurement unit for carrying out a measurement on a sample, a microprocessor for controlling the measurement unit, a display for displaying measurement results, and at least one operating element via which a user can input data and/or control commands, whereby the handheld analysis device is configured in such a manner that actuation of the at least one operating element can put the device in a selection mode, in which the device can be put, by means of the operating element(s), in one of several setting modes, in which a user can change device settings by inputting data and/or control commands, whereby any one of the setting modes is always selected in the selection mode, such that the device can be put in the selected setting mode by means of the operating element(s), and whereby it can be set in the selection mode, which setting mode is selected, by actuating the operating element(s).

In order to change a device setting in the handheld analysis device according to embodiments of the invention, it is no longer necessary to consecutively flip through various device settings to reach other device functions which usually do not need to be changed anyway. In a handheld analysis device according to the invention, a specific setting mode is provided for changing each of the various device settings and the setting mode can be activated specifically in order to change the respective device settings and adapt the corresponding device functions to a personal user profile. Accordingly, for example the time, limit values or reminder times at which a user is to be alerted by means of a signal to carry out a measurement, can be set independent of each other.

For changing a device setting, the device is first put in the selection mode. In the selection mode, the device can be put in a selected setting mode by actuating an operating element. In order to simplify the operation, it is advantageous to assign to the individual setting modes symbols (e.g. F1, F2, F3, etc.) that can be displayed on the display. Preferably, one of the setting modes is always displayed as selected setting mode in the selection mode, for example by displaying or highlighting the symbol representing the respective setting mode (e.g. F1) on the display. If the user wishes to put the device in the selected setting, mode, the selection thus made can be confirmed by actuating an operating element such that the device is put in the selected setting mode, meaning that the selected setting mode is being activated. When the device is in the selection mode and the user wishes to use a setting mode different from the selected one, he can change the selected setting mode by actuating an operating element. As soon as the desired setting mode is selected, the user can put the device in this setting mode.

### BRIEF DESCRIPTION OF THE DRAWINGS

Further details and advantages of the invention shall be illustrated by means of an exemplary embodiment and by making reference to the appended drawings. The particularities shown therein can be used alone or in combination in order to create preferred developments of the invention. In the figures:

FIG. 1 shows a flow diagram detailing the steps involved in changing a device setting of an exemplary embodiment of a handheld analysis device according to the invention; and,

FIG. 2 shows an exemplary embodiment of a display of a handheld analysis device according to the invention.

### DETAILED DESCRIPTION

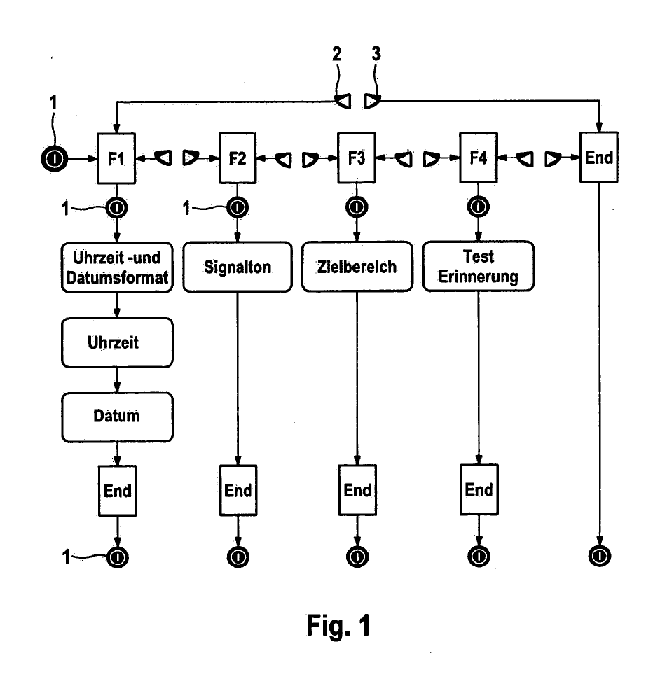

FIG. 1 illustrates in a flow diagram the steps that need to be carried out to change a device setting of a handheld analysis device according to the invention. An exemplary embodiment of a handheld analysis device according to the invention is not shown, since the visible components thereof correspond to those of commercial handheld analysis devices.

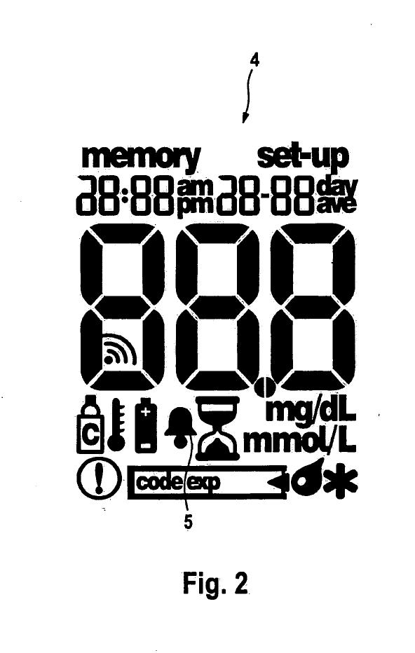

The handheld analysis device for examining a sample of a human or animal body fluid has a measuring unit for carrying out a measurement on a sample, a microprocessor for controlling the measuring unit, a display 4 shown in FIG. 2 for displaying measurement results, an acoustic signal device which, in operation, generates an acoustic reminder signal at predetermined points in time as a reminder of carrying out a measurement, and three operating elements 1, 2, 3 that are shown in conjunction with arrows in various places in the flow diagram shown in FIG. 1. Actuation of the respective operating element 1, 2, 3 leads to the next position in the flow diagram in the direction of the arrow. The operating elements 1, 2, 3 can be keys such as keys for five operating elements 1, 2, 3, or such as key for three operating elements 1, 2, 3.

When the operating element 1 is actuated by being pressed for at least three seconds, the handheld analysis device transitions to a selection mode in which the device can be put, by means of the operating elements 1, 2, 3, in one of several setting modes F1, F2, F3, F4, in which a user can change device settings by inputting data and/or control commands. When the device is in the selection mode, any one of the setting modes F1, F2, F3, F4 can be selected.

Initially, setting mode F1 is selected by default after activation of the selection mode in the exemplary embodiment shown. By actuating the operating element 1, the device is put in the currently selected setting mode. According to the flow diagram shown in FIG. 1, the handheld analysis device therefore transitions to the selection mode upon sustained pressing of the operating element 1, whereby the setting mode F1 is selected initially by default. By actuating the operating element 1 again, the device is put in the selected setting mode (e.g. F1). In setting mode F1, a time of day and a date can be input in common manner by means of the operating elements 1, 2, 3 in order to set a clock that is integrated in the device and in order to facilitate display of the date. For this reason, the first setting mode F1 is called the time setting mode. After the time and date are set, actuating the operating element 1 again, the handheld analysis device is put in an operating or resting mode.

In order to change another device setting, the operating elements 2, 3 can be actuated in the selection mode to change the setting mode F1, F2, F3, F4 that is being selected. In this context, actuating the operating element 3 leads to the next higher setting mode being selected from a list of setting modes F1, F2, F3, F4 that is stored in the device. In simultaneous manner, actuating the operating element 2 leads to the selection of the corresponding preceding setting mode F1, F2, F3, F4 from the list.

If the first setting mode F1 of the list is selected, actuating the operating element 2 leads to exiting from the selection mode, which is shown in FIG. 1 by the symbol, “End”. Actuating the operating element 1 then causes the device to transition to an operating or resting mode. Accordingly, actuating the operating element 3 leads to exiting from the selection mode if the last setting mode F4 of the list is selected.

The second setting mode F2 serves to put the device in an acoustic mode, in which measurement results can be displayed by means of acoustic signals for people with impaired vision. For this reason, the second setting mode F2 is called acoustic setting mode. Acoustic user guidance can be activated in the second setting mode F2, in which acoustic information, in particular in the form of speech, is put out as an operating aid. In order to activate the acoustic user guidance, the ON1 state needs to be set in setting mode F2. In addition, the acoustic mode can also be activated completely in setting mode F2 such that measurement results are also put out acoustically by setting the ON2 state. If no acoustic output is desired, the OFF state can be set in setting mode F2 such that there is neither an acoustic output of measurement results nor acoustic user guidance.

If the third setting mode F3 is activated, limit values can be programmed that can be used by the microprocessor to compare measurement values, that are to be determined by the measurement unit, to and to actuate the signal device if a measurement value exceeds an upper limit value or fails to reach a lower limit value such that an acoustic warning signal is generated by the signal device if either case occurs. For this reason, the third setting mode F3 is called limit value setting mode.

If the handheld analysis device is in the fourth setting mode F4, reminder times can be programmed at which the signal device is actuated by the microprocessor and generates a reminder signal in order to remind a user to carry out a measurement. For this reason, the fourth setting mode F4 is called alarm clock setting mode.

Each of the setting modes F1, F2, F3, F4 has a symbol assigned to that is displayed on display 4 when the device is in the selection mode and the respective setting mode F1, F2, F3, F4 is selected. Particularly well-suited for use as symbols for the various setting modes are combinations of a letter and a number since symbols of this type can be shown with little effort on an LCD display 4, in particular, symbols of this type can be shown on a reasonably-priced segment display 4 as the one shown in FIG. 2.

Although a segment display 4 having a fixed segment display has fewer graphical options as compared to a dot matrix, it is also substantially more reasonably-priced. The segment display 4 can be configured for displaying not only measurement results and symbols of the setting modes F1, F2, F3, F4, but also simple graphical symbols as operating aid, for example a bell 5 symbolizing that the reminder function is activated and a reminder time is set.

The handheld analysis device can be operated independently from the power grid and is provided, for this purpose, with an electrical grid independent power supply, for example batteries of solar cells.

Thus, embodiments of the hand-held analysis device having different setting modes are disclosed. One skilled in the art will appreciate that the teachings can be practiced with embodiments other than those disclosed. The disclosed embodiments are presented for purposes of illustration and not limitation, and the invention is only limited by the claims that follow.

What is claimed is:

1. A handheld analysis device for examining a sample of a human or animal body fluid, comprising:
a measurement unit for carrying out a measurement on a sample;
a microprocessor for controlling the measurement unit;
a segment display for displaying measurement results, and
at least one operating element for a user to input data and/or control commands,
whereby the handheld analysis device is configured in such a manner that actuation of the operating element places the device in a selection mode and further actuation of the operating element places the device in one of several setting modes in which a user can change device settings by inputting data and/or control commands,
whereby any one of the setting modes is always selected in the selection mode, such that the device can be put in the selected setting mode by means of the at least one operating element, and
whereby the setting mode that is selected is selected in the selection mode by actuating the operating element.

2. The handheld analysis device according to claim 1, wherein one of the setting modes is a time setting mode in which a time of day is inputted in order to set a clock that is integrated in the device.

3. The handheld analysis device according to claim 1, further comprising,
a signal device that generates a reminder signal at times that are set in order to remind a user to carry out a measurement.

4. The handheld analysis device according to claim 3, wherein one of the setting modes is an alarm clock setting mode, in which reminder times are programmed at which the signal device is actuated by the microprocessor and generates a reminder signal in order to remind a user to carry out a measurement.

5. The handheld analysis device according to claim 1 wherein one of the setting modes is a limit value setting mode in which limit values are programmed that are used by the microprocessor to compare measurement values that are to be determined by the measurement unit to actuate the signal device if a measurement value exceeds an upper limit value or falls below a lower limit value such that an acoustic warning signal is generated by the signal device if either case occurs.

6. The handheld analysis device according to claim 1, wherein one of the setting modes is an acoustic setting mode, in which an acoustic mode can be activated, in which measurement results are displayed by acoustic signals or an acoustic user guidance is provided.

7. The handheld analysis device according to claim 1, wherein the handheld analysis device is configured in such a manner that actuation of a first operating element places the device in the selection mode, and actuation of the first operating element or a second operating element places the device in one of the setting modes.

8. The handheld analysis device according to claim 7, wherein the selection mode is placed in the selected setting mode by actuating the first operating element.

9. The handheld analysis device according to claim 8, wherein the setting mode is set in the selection mode by actuating the second operating element.

10. The handheld analysis device according to claim 1, wherein each setting mode has a symbol assigned to it that is displayed on the display when the device is in the selection mode and the corresponding setting mode is selected.

11. The handheld analysis device according to claim 1, wherein the first and the second operating element are keys.

12. The handheld analysis device according to claim 1, wherein the signal device is an acoustic signal device for generating acoustic signals.

13. The handheld analysis device according to claim 1, wherein there are from one to five operating elements.

14. The handheld analysis device according to claim 1 wherein it can be operated independently of an electrical grid.

15. A handheld analysis device for examining a sample of a human or animal body fluid, comprising:
a measurement unit for carrying out a measurement on a sample;
a microprocessor for controlling the measurement unit;
a segment display for displaying measurement results, and
at least one operating element via which a user inputs data and/or control commands in a handheld analysis device,
means for selecting a mode with actuation of the operating element places the device in a selection mode and further actuation of the operating element places the device in one of several setting modes, in which a user can change device settings by inputting data and/or control commands,

16. The handheld analysis device according to claim 15 wherein one of the setting modes is a limit value setting mode, in which limit values are programmed that are used by the microprocessor to compare measurement values, that are to be determined by the measurement unit, to and to actuate the signal device if a measurement value exceeds an upper limit value or falls below a lower limit value such that an acoustic warning signal is generated by the signal device if either case occurs.
